(19) United States
(12) Patent Application Publication
(10) Pub. No.: US 2023/0330001 A1
(43) Pub. Date: Oct. 19, 2023

(54) AGENT FOR IMPROVING SKIN CONDITION

(71) Applicant: MITSUI SUGAR CO.,LTD., Tokyo (JP)

(72) Inventor: Kohei Miyazaki, Shinjuku-ku, Tokyo (JP)

(73) Assignee: Mitsui DM Sugar Co.,Ltd., Tokyo (JP)

(21) Appl. No.: 18/026,937

(22) PCT Filed: Jan. 6, 2022
(86) PCT No.: PCT/JP2022/000260

§ 371 Date: Mar. 17, 2023

(30) Foreign Application Priority Data
Mar. 9, 2021 (JP) 2021-037382

(51) Int. Cl. A61K 8/97; A61Q 19/08

(52) U.S. Cl. CPC A61K 8/97; A61Q 19/08

(57) ABSTRACT

The present invention relates to an agent for improving skin condition, including: a bagasse-derived polyphenol composition as an active ingredient.

### TECHNICAL FIELD

The present invention relates to an agent for improving skin condition.

### BACKGROUND ART

In recent years, the utilization of biomass derived from natural products has been studied from the viewpoint of reducing environmental loads, and research thereon is being conducted in various fields. For example, Patent Literature 1 discloses a method for producing a polyphenol composition from bagasse, the method including: pretreating bagasse using at least one alkaline solution selected from the group consisting of an aqueous sodium hydroxide solution, an aqueous potassium hydroxide solution, and an aqueous ammonia solution to obtain a pretreatment solution; adjusting a pH of the pretreatment solution to be acidic with hydrochloric acid, and then filtering the pretreatment solution to recover a filtrate; and passing the filtrate through a column filled with an aromatic synthetic adsorbent and eluting a component adsorbed onto the aromatic synthetic adsorbent with a mixed solvent of ethanol and water to obtain an eluted fraction as the polyphenol composition.

### CITATION LIST

### Patent Literature

[Patent Literature 1] Japanese Unexamined Patent Publication No. 2019-206489

### SUMMARY OF INVENTION

### Technical Problem

An object of the present invention is to provide a new application for a bagasse-derived polyphenol composition for more effective utilization of biomass.

### Solution to Problem

The present inventors have found that a bagasse-derived polyphenol composition has an effect of improving skin smoothness, an effect of reducing skin wrinkles, an effect of suppressing skin pigmentation, and an effect of increasing skin moisture content. The present invention is based on these findings.

As one aspect, the present invention provides an agent for improving skin condition including: a bagasse-derived polyphenol composition as an active ingredient. Since the bagasse-derived polyphenol composition has an effect of improving skin smoothness, an effect of reducing skin wrinkles, an effect of suppressing skin pigmentation, and an effect of increasing skin moisture content as described above, the agent for improving skin condition according to the present invention can improve skin condition based on at least one effect selected from the effect of improving skin smoothness, the effect of reducing skin wrinkles, the effect of suppressing skin pigmentation, and the effect of increasing skin moisture content.

The above-described bagasse-derived polyphenol composition may be obtained through a method including: a step of pretreating bagasse using at least one alkaline solution selected from the group consisting of an aqueous sodium hydroxide solution, an aqueous potassium hydroxide solution, and an aqueous ammonia solution to obtain a pretreatment solution; a step of adjusting a pH of the above-described pretreatment solution to be acidic with hydrochloric acid, and then filtering the pretreatment solution to recover a filtrate; and a step of passing the above-described filtrate through a column filled with an aromatic synthetic adsorbent and eluting components adsorbed onto the above-described aromatic synthetic adsorbent with a mixed solvent of ethanol and water to obtain an eluted fraction as the polyphenol composition. Accordingly, the effect of improving skin smoothness, the effect of reducing skin wrinkles, the effect of suppressing skin pigmentation, and the effect of increasing skin moisture content are exhibited more effectively.

A temperature of the above-described alkaline solution may be 60° C. to 100° C.

The above-described alkaline solution may be an aqueous sodium hydroxide solution.

A concentration of the aqueous sodium hydroxide solution may be 0.1 to 1.0% by mass.

The above-described aromatic synthetic adsorbent may be one consisting of a styrene-divinylbenzene resin.

The agent for improving skin condition according to the present invention may be for use in improving skin smoothness, reducing skin wrinkles, suppressing skin pigmentation, or increasing skin moisture content.

As another aspect, the present invention provides an agent for improving skin smoothness, an agent for reducing skin wrinkles, an agent for suppressing skin pigmentation, or an agent for increasing skin moisture content which contain a bagasse-derived polyphenol composition as an active ingredient.

### Advantageous Effects of Invention

According to the present invention, it is possible to provide a new application for a bagasse-derived polyphenol composition.

### BRIEF DESCRIPTION OF DRAWINGS

FIG. 1 is a graph showing evaluation results of smoothness (SEsm).

FIG. 2 is a graph showing evaluation results of wrinkles (SEw).

FIG. 3 is a graph showing evaluation results of melanin levels.

FIG. 4 is a graph showing evaluation results of transepidermal water loss.

FIG. 5 is a graph showing evaluation results of facial epidermis moisture content.

### DESCRIPTION OF EMBODIMENTS

Hereinafter, an embodiment of the present invention will be described in detail. However, the present invention is not limited to the following embodiment.

An agent for improving skin condition according to the present embodiment contains a bagasse-derived polyphenol composition as an active ingredient. Since the bagasse-derived polyphenol composition has an action of improving skin smoothness, an action of reducing skin wrinkles, an action of suppressing skin pigmentation, and an action of increasing skin moisture content, the agent for improving skin condition according to the present invention can improve skin condition based on at least one action selected from the action of improving skin smoothness, the action of reducing skin wrinkles, the action of suppressing skin pigmentation, and the action of increasing skin moisture content.

Improving skin condition includes cosmetically improving skin condition. Improving skin condition includes rejuvenating skin condition, keeping skin condition youthful, and the like. More specifically, improving skin condition includes, for example, improving skin smoothness, reducing skin wrinkles, suppressing skin pigmentation, and increasing skin moisture content. Accordingly, the agent for improving skin condition according to the present embodiment can be used, for example, for anti-aging.

The bagasse-derived polyphenol composition which is an active ingredient is a composition which contains one or more polyphenols and is obtained through processing such as extraction, purification, and/or isolation of bagasse as a raw material. Polyphenols in the present specification are phenolic compounds that can be measured through the Folin-Ciocalteu method. More specifically, the polyphenols may be phenylpropanoids such as p-coumaric acid or ferulic acid, flavonoids such as catechin or anthocyanin, and the like.

In the present specification, bagasse is sugar cane pomace and typically refers to bagasse discharged in a sugar production process in a sugar factory. Bagasse discharged in a sugar production process in a sugar factory includes not only final bagasse that has exited a final compressor but also shredded sugar cane bitten in the subsequent compressor including a first compressor. Bagasse discharged after sugar juice has been compressed out through a compression step in a sugar factory is preferably used. Bagasse discharged from the compression step has different moisture contents, sugar contents, and compositional ratios depending on the type of sugar cane, harvest time, and the like, and can be used arbitrarily. Bagasse may be bagasse remaining after compressing sugar cane discharged in a brown sugar factory. In addition, in a small-scale implementation at a laboratory level, bagasse, after a sugar solution has been compressed out from sugar cane, may be used.

In one embodiment, the bagasse-derived polyphenol composition may be obtained through a method including: a step of pretreating bagasse using at least one alkaline solution selected from the group consisting of an aqueous sodium hydroxide solution, an aqueous potassium hydroxide solution, and an aqueous ammonia solution to obtain a pretreatment solution (pretreatment step); a step of adjusting a pH of the obtained pretreatment solution to be acidic with hydrochloric acid, and then filtering the pretreatment solution to recover a filtrate (filtration step); and a step of passing the above-described filtrate through a column filled with an aromatic synthetic adsorbent and eluting components adsorbed onto the aromatic synthetic adsorbent with a mixed solvent of ethanol and water to obtain an eluted fraction as the polyphenol composition (elution step).

The pretreatment step may be processing of bringing the alkaline solution into contact with bagasse in one embodiment. Examples of the method for bringing an alkaline solution into contact therewith include a method for sprinkling an alkaline solution on bagasse and a method for immersing bagasse in an alkaline solution. In the method for immersing bagasse in an alkaline solution, bagasse may be immersed while stirring a mixture of bagasse and an alkaline solution.

The alkaline solution may be at least one selected from the group consisting of an aqueous sodium hydroxide solution, an aqueous potassium hydroxide solution, and an aqueous ammonia solution. The alkaline solution is preferably an aqueous sodium hydroxide solution from the viewpoint of being inexpensive.

The concentration of an alkaline solution may be appropriately set according to the type of alkaline solution to be used, but is preferably higher than or equal to 0.1% by mass, more preferably higher than or equal to 0.2% by mass, and still more preferably higher than or equal to 0.3% by mass from the viewpoint of shortening the processing time taken for pretreatment. The concentration of an alkaline solution is preferably lower than or equal to 10% by mass, more preferably lower than or equal to 5% by mass, and still more preferably lower than or equal to 1.0% by mass from the viewpoint of improving extraction efficiency.

An alkaline solution in the pretreatment step is preferably heated. The temperature (liquid temperature) of an alkaline solution is preferably higher than or equal to 50° C., more preferably higher than or equal to 60° C., and still more preferably higher than or equal to 80° C. from the viewpoint of shortening the processing time taken for pretreatment. The temperature of an alkaline solution is preferably lower than or equal to 110° C., more preferably lower than or equal to 105° C., and still more preferably lower than or equal to 100° C. from the viewpoint of preventing polysaccharides from remaining in a pretreatment solution.

The amount of alkaline solution added may be greater than or equal to 50 parts by mass, greater than or equal to 100 parts by mass, or greater than or equal to 1,000 parts by mass with respect to 100 parts by mass of bagasse. In addition, the processing time in the pretreatment step may be appropriately adjusted according to the type, temperature, and addition amount of the alkaline solution, and may be, for example, 1 to 5 hours.

The pH of a pretreatment solution may be greater than or equal to 8, or greater than or equal to 9 and less than or equal to 13, or less than or equal to 12.

In the pretreatment step according to the present embodiment, insoluble components may be separated from liquid components after performing the above-described alkali treatment. In this case, the separated liquid components can be used as a pretreatment solution. The method for separating insoluble components from liquid components may include separation performed using a strainer and through filtration, centrifugation, decantation, and the like.

Next, the pH of the pretreatment solution obtained in the pretreatment step is adjusted to be acidic with hydrochloric acid, and the pretreatment solution is filtered to recover a filtrate (filtration step).

In the filtration step, hydrochloric acid is first added to the pretreatment solution to adjust the pH of the pretreatment solution to be acidic. The concentration of hydrochloric acid may be appropriately set as long as the pH of the pretreatment solution can be adjusted, and may be, for example, 0.1 to 35% by mass.

The pH of the pretreatment solution (hereinafter, also referred to as an “acidic pretreatment solution”) after the addition of hydrochloric acid is preferably greater than or equal to 1.5, more preferably greater than or equal to 2.0, and still more preferably greater than or equal to 2.5, and preferably less than or equal to 4.5, more preferably less than or equal to 4.0, and still more preferably less than or equal to 3.5 from the viewpoint of achieving both suppression of coagulative precipitation of polyphenols and adsorption of a synthetic adsorbent. In a case where the pH of the acidic pretreatment solution is greater than or equal to 1.5, polyphenols are less likely to be coagulated and precipitated. Therefore, polyphenols are less likely to be removed through filtration even if the filtration is performed after pH adjustment. On the other hand, in a case where the pH of the acidic pretreatment solution is less than or equal to 4.5, polyphenols can be made to be easily adsorbed onto an aromatic synthetic adsorbent in an elution step to be described below. That is, in the case where the pH of the acidic pretreatment solution is within the above-described ranges, it is possible to promote adsorption onto an aromatic synthetic adsorbent while suppressing coagulative precipitation of polyphenols.

In the case where the pH of the acidic pretreatment solution is adjusted to within the above-described ranges, insoluble components are precipitated in the acidic pretreatment solution. In the filtration step, the precipitated insoluble components are removed through filtration. For the filtration, natural filtration, vacuum filtration, pressure filtration, centrifugal filtration, and the like may be performed, and pressure filtration is preferably performed. Pressure filtration may be performed using a pressure filter (filter press).

A filter aid may be added to the acidic pretreatment solution at the time of filtration. Examples of filter aids include diatomaceous earth, perlite, and cellulose. In the case where a filter aid is added, the content of the filter aid may be 0.2 to 2.0% by mass based on the total amount of the acidic pretreatment solution.

Next, the filtrate obtained in the filtration step is passed through a column filled with an aromatic synthetic adsorbent. Components adsorbed onto the aromatic synthetic adsorbent are eluted with a mixed solvent of ethanol and water, and eluted fractions are obtained to enable production of a polyphenol composition (elution step).

The aromatic synthetic adsorbent is a synthetic adsorbent consisting of an aromatic resin from the viewpoint of efficiently adsorbing a polyphenol composition contained in the filtrate. A styrene-divinylbenzene aromatic resin is preferable as an aromatic resin. Examples of the styrene-divinylbenzene aromatic resin include a porous resin such as an aromatic resin having a hydrophobic substituent, an unsubstituted aromatic resin, and an aromatic resin obtained by subjecting an unsubstituted aromatic resin to a special treatment. As the styrene-divinylbenzene aromatic resin, an unsubstituted aromatic resin or an aromatic resin obtained by subjecting an unsubstituted aromatic resin to a special treatment of increasing a specific surface area is preferable and an aromatic resin obtained by subjecting an unsubstituted aromatic resin to a special treatment of increasing a specific surface area is more preferable.

A specific surface area of the aromatic synthetic adsorbent is, as dry mass, preferably greater than or equal to 500 m²/g and more preferably 700 m²/g from the viewpoint of improving the adsorption rate. The specific surface area of the aromatic synthetic adsorbent can be calculated by applying a measured value of a gas adsorption method to a BET equation. The most frequent pore diameter of the aromatic synthetic adsorbent is preferably less than or equal to 600 Å, more preferably less than or equal to 300 Å, and still more preferably 200 Å from the viewpoints of high separation performance and high adsorption performance. The most frequent pore diameter can be measured through the gas adsorption method.

Such a synthetic adsorbent is commercially available, and examples thereof include DIAION (trademark) HP-10, HP-20, HP-21, HP-30, HP-40, and HP-50 (all of which are trade names of unsubstituted aromatic resins and manufactured by Mitsubishi Chemical Corporation); SP-825, SP-800, SP-850, SP-875, SP-70, and SP-700 (all of which are trade names of aromatic resins obtained by subjecting an unsubstituted aromatic resin to a special treatment and are manufactured by Mitsubishi Chemical Corporation); SP-900 (which is a trade name of an aromatic resin and manufactured by Mitsubishi Chemical Corporation); AMBERLITE (trademark) XAD-2, XAD-4, XAD-16, XAD-18, and XAD-2000 (all of which are trade names of aromatic resins and manufactured by ORGANO CORPORATION); DIAION (trademark) SP-205, SP-206, and SP-207 (all of which are trade names of aromatic resins having a hydrophobic substituent and manufactured by Mitsubishi Chemical Corporation); and HP-2MG and EX-0021 (all of which are trade names of aromatic resins having a hydrophobic substituent and manufactured by Mitsubishi Chemical Corporation). Among these, DIAION (trademark) SP-850 is preferable. These synthetic adsorbents may be used alone or in combination of two or more thereof

The amount of aromatic synthetic adsorbent with which a column is filled can be appropriately determined depending on the size of the column, the type of synthetic adsorbent, and the like.

When a filtrate is passed through a column, the temperature of the filtrate may be 25° C. to 45° C. The liquid passage amount and the liquid passage rate when passing a filtrate through a column can be appropriately determined depending on the type of aromatic synthetic adsorbent, and the like.

In the elution step, the components adsorbed onto the column are eluted with a mixed solvent of ethanol and water after the completion of passing the filtrate. The mixing volume ratio (ethanol:water) of the mixed solvent may be 50:50 to 99:1, and is preferably within a range of 50:50 to 70:30 from this viewpoint improving the elution efficiency. The elution rate can be appropriately determined depending on the size of the column, the type of aromatic synthetic adsorbent, and the like. In order to efficiently elute the components adsorbed onto the column, it is preferable to wash the inside of the column with water before passing the filtrate through the column.

A polyphenol composition derived from bagasse can be produced by obtaining eluted fractions in the elution step.

After the elution step, a step (concentration step) of concentrating eluted fractions (polyphenol composition) may be further provided as necessary. In the concentration step, the eluted fractions may be concentrated, for example, 5 to 20 fold using a centrifugal thin-film vacuum evaporator. Accordingly, a concentrated liquid containing a polyphenol composition can be obtained.

The polyphenol composition that can be produced through the method of the present embodiment may mainly contain coumaric acid and ferulic acid. The fact that the polyphenol composition contains polyphenols can be confirmed by measuring a polyphenol content through the Folin-Ciocalteu method. In addition, the composition of the polyphenol composition can be confirmed by qualitatively and quantitatively analyzing the obtained polyphenol composition through high-performance liquid chromatograph (HPLC).

The agent for improving skin condition according to the present embodiment can be used, for example, as cosmetics, pharmaceuticals, or quasi-drugs.

The agent for improving skin condition according to the present embodiment may consist of only a bagasse-derived polyphenol composition which is an active ingredient, or materials that can be used in cosmetics, quasi-drugs, or pharmaceutical products may also be incorporated. The materials that can be used in cosmetics, quasi-drugs, or pharmaceuticals are not particularly limited, and examples thereof include oil agents, surfactants, moisturizing components, thickener, powders, inorganic pigments, organic dyes, organic powders, ultraviolet absorbers, lower alcohols, vitamins, proteins, and lipids.

Examples of oil agents include oils, hydrocarbons, higher fatty acids, higher alcohols, ester oils, and silicone oils. In addition, examples of oils include mineral oil, macadamia nut oil, avocado oil, corn oil, olive oil, rapeseed oil, sesame oil, castor oil, safflower oil, cottonseed oil, jojoba oil, palm oil, liquid lanolin, hydrogenated coconut oil, hydrogenated oil, Japan wax, hydrogenated castor oil, beeswax, candelilla wax, carnauba wax, insect wax, lanolin, reduced lanolin, hard lanolin, and jojoba wax. Examples of hydrocarbons include hydrocarbons such as waxes, liquid paraffin, squalane, pristane, ozokerite, paraffin, ceresin, vaseline, and microcrystalline wax. Examples of higher fatty acids include oleic acid, isostearic acid, lauric acid, myristic acid, palmitic acid, stearic acid, behenic acid, and undecylenic acid. Examples of higher alcohols include cetyl alcohol, stearyl alcohol, isostearyl alcohol, behenyl alcohol, octyldodecanol, myristyl alcohol, and cetostearyl alcohol. Examples of ester oils include cetyl isooctanoate, isopropyl myristate, hexyldecyl isostearate, diisopropyl adipate, di-2-ethylhexyl sebacate, cetyl lactate, diisostearyl malate, ethylene glycol di-2-ethylhexanoate, neopentyl glycol dicaprate, glycerol di-2-heptylundecanoate, glycerol tri-2-ethylhexanoate, trimethylolpropane tri-2-ethylhexanoate, trimethylolpropane triisostearate, pentaerythritol tetra-2-ethylhexanoate, and alkyl benzoate. Examples of silicone oils include: linear polysiloxanes such as dimethylpolysiloxane, methylphenylpolysiloxane, and diphenylpolysiloxane; cyclic polysiloxanes such as octamethylcyclotetrasiloxane, decamethylcyclopentasiloxane, and dodecamethylcyclohexanesiloxane; modified polysiloxanes such as amino-modified polysiloxane, polyether-modified polysiloxane, alkyl-modified polysiloxane, and fluorine-modified polysiloxane.

Examples of surfactants include anionic surfactants, cationic surfactants, amphoteric surfactants, and nonionic surfactants. In addition, examples of anionic surfactants include fatty acid soap (such as sodium laurate and sodium palmitate), potassium lauryl sulfate, and triethanolamine ether alkyl sulfate. Examples of cationic surfactants include stearyltrimethylammonium chloride, benzalkonium chloride, and laurylamine oxide. Examples of amphoteric surfactants include imidazoline amphoteric surfactants (such as 2-cocoyl-2-imidazolinium hydroxide-1-carboxyethyloxy disodium salt), betaine surfactants (such as alkylbetaine, amide betaine, and sulfobetaine), and acylmethyl taurine. Examples of nonionic surfactants include sorbitan fatty acid esters (such as sorbitan monostearate and sorbitan sesquioleate), glycerol fatty acids (such as glycerol monostearate), propylene glycol fatty acid esters (such as propylene glycol monostearate), hydrogenated castor oil derivatives, glycerol alkyl ethers, POE sorbitan fatty acid esters (POE sorbitan monooleate, POE sorbitan trioleate, and polyoxyethylene sorbitan monostearate), POE sorbitol fatty acid esters (such as POE-sorbitol monolaurate), POE glycerol fatty acid esters (such as POE-glycerol monoisostearate), POE fatty acid esters (such as polyethylene glycol monooleate and POE distearate), POE alkyl ethers (such as POE 2-octyldodecyl ethers), POE alkyl phenyl ethers (such as POE nonylphenyl ethers), Pluronic (registered trademark) types, POE·POP alkyl ethers (such as POE·POP 2-decyltetradecyl ether), Tetronics, POE castor oil·hydrogenated castor oil derivatives (such as POE castor oil and POE hydrogenated castor oil), sucrose fatty acid esters, and alkyl glucosides.

Examples of moisturizing components include: polyhydric alcohols such as polyethylene glycol, glycerol, 1,3-butylene glycol, erythritol, sorbitol, xylitol, maltitol, propylene glycol, dipropylene glycol, diglycerol, isoprene glycol, 1,2-pentanediol, 2,4-hexylene glycol, 1,2-hexanediol, and 1,2-octanediol; sodium pyrrolidone carboxylate; lactic acid; and sodium lactate.

Examples of thickeners include guar gum, quince seed, carrageenan, galactan, gum arabic, pectin, mannan, starch, xanthan gum, curdlan, methyl cellulose, hydroxyethyl cellulose, carboxymethyl cellulose, methyl hydroxypropyl cellulose, chondroitin sulfate, dermatan sulfate, glycogen, heparan sulfate, hyaluronate, sodium hyaluronate, tragacanth gum, keratan sulfate, chondroitin, mucoitin sulfate, hydroxyethyl guar gum, carboxymethyl guar gum, dextran, keratosulfate, locust bean gum, succinoglucan, caronic acid, chitin, chitosan, carboxymethyl chitin, agar, polyvinyl alcohol, polyvinyl pyrrolidone, carboxyvinyl polymer, sodium polyacrylate, polyethylene glycol, an acrylic acid/acrylamide copolymer, and bentonite.

Examples of powders include mica, talc, kaolin, synthetic mica, calcium carbonate, magnesium carbonate, silicic anhydride (silica), aluminum oxide, and barium sulfate. Their surfaces may be treated.

Examples of inorganic pigments include red oxide, yellow iron oxide, black iron oxide, cobalt oxide, ultramarine blue, iron blue, titanium oxide, and zinc oxide. Their surfaces may be treated.

Examples of organic dyes include: pearlescent agents such as mica titanium, fish scale flake, and bismuth oxychloride; and Red No. 202, Red No. 228, Red No. 226, Yellow No. 4, Blue No. 404, Yellow No. 5, Red No. 505, Red No. 230, Red No. 223, Orange No. 201, Red No. 213, Yellow No. 204, Yellow No. 203, Blue No. 1, Green No. 201, Violet No. 201, and Red No. 204 which may be laked. Their surfaces may be treated.

Examples of organic powders include polyethylene powder, polymethyl methacrylate, nylon powder, and an organopolysiloxane elastomer.

Examples of ultraviolet absorbers include para-aminobenzoic acid ultraviolet absorbers, anthranilic acid ultraviolet absorbers, salicylic acid ultraviolet absorbers, cinnamic acid ultraviolet absorbers, benzophenone ultraviolet absorbers, sugar ultraviolet absorbers, 2-(2′-hydroxy-5′-t-octylphenyl)benzotriazole, and 4-methoxy-4′ -t-butyldibenzoylmethane.

Examples of lower alcohols include lower alcohols such as ethanol and isopropanol.

Examples of vitamins include: vitamin A or derivatives thereof; vitamin Bs such as vitamin B6 hydrochloride, vitamin B6 tripalmitate, vitamin B6 dioctanoate, vitamin B2, or derivatives thereof, and vitamin B12, vitamin B15, or derivatives thereof; vitamin Es such as α-tocopherol, β-tocopherol, γ-tocopherol, and vitamin E acetate; vitamin Ds; vitamin H; pantothenic acid; pantethine; and pyrroloquinoline.

Examples of proteins include plant-derived proteins such as wheat protein, soybean protein, and soybean isoflavone; animal-derived proteins such as keratin, keratin hydrolysate, sulfone-based keratin, lactoferrin, collagen, and elastin; derivatives thereof; and salts thereof

Examples of lipids include: glycerophospholipids such as phosphatidylcholine, phosphatidylethanolamine, phosphatidylserine, phosphatidic acid, phosphatidylglycerol, phosphatidylinositol, cardiolipin, egg yolk lecithin, hydrogenated egg yolk lecithin, soybean lecithin, and hydrogenated soybean lecithin; sphingophospholipids such as sphingomyelin, ceramide phosphorylethanolamine, and ceramide phosphorylglycerol; plasmalogens; glycerolipids in which glycolipids are digalactosyl diglyceride and galactosyl diglyceride sulfate; sphingoglycolipids such as galactosylceramide, galactosylceramide sulfate, lactosylceramide, ganglioside G7, ganglioside G6, and ganglioside G4, and a mixture thereof; monoglycerides; diglycerides; triglycerides; sphingolipids; terpenes; steroids; and prostaglandins.

In a case where the agent for improving skin condition according to the present embodiment is mixed with other materials, the content of bagasse-derived polyphenol composition which is an active ingredient may be appropriately set according to the form of the agent for improving skin condition to be described below and the purpose of use, but is preferably 0.1% by mass or higher, more preferably 0.3% by mass or higher, and still more preferably 0.5% by mass or higher, or preferably 10% by mass or lower, more preferably 5% by mass or lower, and still more preferably 3% by mass or lower, as a solid content, from the viewpoint of more effectively exhibiting the effect of improving skin condition.

The form of the agent for improving skin condition according to the present embodiment is not limited, and the agent for improving skin condition may be in any form of solids (such as powder or granules), liquids (such as solutions or suspensions), and paste, and may be in any dosage form such as powder agents, pills, granules, tablets, capsules, troches, liquid agents, and suspensions.

In a case where the agent for improving skin condition according to the present embodiment is used as cosmetics, the cosmetics may include basic cosmetic materials such as toner, milky lotion, lotion, cream, serums, oil, pack, and lip balm, hairdressing agents such as a hair tonic and a hair liquid, hair cosmetics such as a hair growing/nourishing agent, and makeup cosmetics such as foundation, lipstick, cheek rouge, eye shadow, eyeliner, mascara, and eyebrow liner.

The agent for improving skin condition according to the present embodiment is preferably administered to be applied to the skin. The bagasse-derived polyphenol composition is applied to the skin so that the application amount per application is preferably 0.5 ng/cm² or more, more preferably 1 ng/cm² or more, and still more preferably 3 ng/cm² or more. In addition, the bagasse-derived polyphenol composition is applied so that the application amount per day is preferably 1 ng/cm² or more, more preferably 2 ng/cm² or more, and still more preferably 6 ng/cm² or more. In addition, the bagasse-derived polyphenol composition is applied so that the application amount per application is preferably 50 ng/cm² or less, more preferably 30 ng/cm² or less, and still more preferably 10 ng/cm² or less. In addition, the bagasse-derived polyphenol composition is applied so that the application amount per day is preferably 100 ng/cm² or less, more preferably 60 ng/cm² or less, and still more preferably 20 ng/cm² or less. Within these ranges, the effect of improving skin condition can be more efficiently exhibited.

The agent for improving skin condition according to the present embodiment has an action of improving skin smoothness, an action of reducing skin wrinkles, an action of suppressing skin pigmentation, and an action of increasing skin moisture content, and therefore can also be used, for example, for improving skin smoothness, reducing skin wrinkles, suppressing skin pigmentation, and increasing skin moisture content.

The above-described present invention can also be regarded as an agent for improving skin smoothness, an agent for reducing skin wrinkles, an agent for suppressing skin pigmentation, or an agent for increasing skin moisture content which contain a bagasse-derived polyphenol composition as an active ingredient. As specific embodiments of the agent for improving skin smoothness, the agent for reducing skin wrinkles, the agent for suppressing skin pigmentation, and the agent for increasing skin moisture content, the specific embodiment described for the agent for improving skin condition can be applied.

The above-described present invention can also be regarded as use of the bagasse-derived polyphenol composition for improving skin condition. The above-described present invention can also be regarded as use of the bagasse-derived polyphenol composition for improving skin smoothness, reducing skin wrinkles, suppressing skin pigmentation, and increasing skin moisture content. As specific embodiments in the aspect, the specific embodiment described for the agent for improving skin condition can be applied.

The above-described present invention can also be regarded as the bagasse-derived polyphenol composition for use in improving skin condition. The above-described present invention can also be regarded as the bagasse-derived polyphenol composition for use in improving skin smoothness, reducing skin wrinkles, suppressing skin pigmentation, and increasing skin moisture content. As specific embodiments in the aspect, the specific embodiment described for the agent for improving skin condition can be applied.

The above-described present invention can also be regarded as use (application) of the bagasse-derived polyphenol composition for producing an agent for improving skin condition. The above-described present invention can also be regarded as use (application) of the bagasse-derived polyphenol composition for producing an agent for improving skin smoothness, an agent for suppressing skin pigmentation, and an agent for increasing skin moisture content. As specific embodiments in the aspect, the specific embodiment described for the agent for improving skin condition can be applied.

The above-described present invention can also be regarded as a method for improving skin condition, the method including administering an agent for improving skin condition to a subject (for example, mammals such as humans) in need thereof The above-described present invention can also be regarded as a method for improving skin smoothness, a method for reducing skin wrinkles, a method for suppressing skin pigmentation, and a method for increasing skin moisture content, the methods including administering an agent for improving skin smoothness, an agent for reducing skin wrinkles, an agent for suppressing skin pigmentation, and an agent for increasing skin moisture content to a subject (for example, mammals such as humans) in need thereof As specific embodiments in the aspect, the specific embodiment described for the agent for improving skin condition can be applied.

### EXAMPLES

Hereinafter, the present invention will be described in more detail based on examples. However, the present invention is not limited to the following examples.

### <Production of Bagasse-Derived Polyphenol Composition>

### (Pretreatment Step)

Pretreatment was performed by adding 3.2 kg of bagasse (with a moisture content of 50% by mass) which is sugar cane pomace and 20 L of a 0.5% (w/w) aqueous sodium hydroxide solution at 90° C. to a cylindrical pot made of stainless steel and mixing them with each other for 2 hours. The pretreated mixed solution was separated into insoluble component and liquid components to obtain about 20 L of the liquid components. This was repeated twice to obtain 40 L of the liquid components (pretreatment solution).

### (Filtration Step)

475 mL of 35% (w/w) hydrochloric acid was added to the total amount of the above-described pretreatment solution, and the pH was adjusted to 3.0. This was regarded as an acidic pretreatment solution. 395 g of diatomaceous earth was added to the acidic pretreatment solution (to become 1% (w/w) based on the pretreatment solution) as a filter aid, and insoluble components were removed by filtering the mixture with a filter press to obtain 38 kg of a filtrate.

### (Elution Step)

The filtrate obtained from the filtration step was passed through a column (column capacity: 1 L) filled with 383 mL of an aromatic synthetic adsorbent (DIAION SP-850 manufactured by Mitsubishi Chemical Corporation) under the condition of a flow rate of 7.6 L/h (SV=20). Thereafter, washing was performed with 10 times as much water as the volume of the synthetic adsorbent, and elution was performed by passing 766 g of a 60% (v/v) aqueous ethanol solution at a SV of 2 to obtain eluted fractions. The eluted fractions were adjusted to have a pH of 6.7 using a 48% (w/w) aqueous sodium hydroxide solution, concentrated to a 10-fold concentration using a rotary evaporator, and then freeze-dried overnight to obtain 23 g of brown powder as a polyphenol composition.

### <Production of Cream>

Components were mixed with each other according to the formulation shown in Table 1 to prepare a cream base. In Table 1, “%” means “w/w%.” A test cream was prepared by adding 1 part by weight of a bagasse-derived polyphenol composition to 100 parts by weight of the prepared cream base and mixing them. In addition, the cream base itself was used as a control cream.

Components used are as follows.

Finsolv Tn (manufactured by Innospec Performance Chemicals Europe Limited): alkyl benzoate

PEMULEN (registered trademark) TR-1 (manufactured by Lubrizol): gelling/thickening agent/polymer, polymer emulsifier

SILSENSE (registered trademark) DW-18 (manufactured by Lubrizol): silicone, emulsifier

NOVEMER (registered trademark) EC-1 (manufactured by Lubrizol): acrylic acid/acrylamide copolymer, mineral oil, POE sorbitan trioleate

GERMABEN (registered trademark) II-E (manufactured by Ashland): bacteriostatic agent

TABLE 1
Composition of cream base

| | | |
|---|---|---|
| A | Finsolv Tn | 25% |
| | PEMULEN (registered trademark) TR-1 | 0.5% |
| B | 1,3-butylene glycol | 1.5% |
| | Glycerol | 1.5% |
| | SILSENSE (registered trademark) DW-18 | 0.5% |
| | Water | 60% |
| C | Triethanolamine (99%) | 0.5% |
| | Water | 5% |
| D | NOVEMER (registered trademark) EC-1 | 4% |
| E | GERMABEN (registered trademark) II-E | 0.5% |

### <Test Method>

A double-blind randomized controlled trial was conducted on 112 healthy subjects aged 18 to 60 (average age: 43.36±10.24). The subjects had no history of allergies to sugar cane extracts, polyphenols, and ferulic acid, had no skin disease, had not undergone laser, Botox, or facial chemical injection within 2 weeks, had not taken immunosuppressants or steroids within 2 weeks, had no immunodeficiency disease, and had given prior informed consent. In addition, the number of subjects (112) was determined with reference to non-patent literature (J. Cosmet. Dermatol., 2020, Vol. 19, pp. 671-676), with a significance level of 95% and power of a test of 90%, and assuming that 20% to 25% of the subjects would drop out.

Both sides of the subjects' faces (left and right sides) were randomly assigned to apply the test cream and the control cream, respectively. Each cream was applied by the subjects themselves twice a day in the morning and evening after cleansing, and this was continued for 4 weeks. The application amount per application was 0.2 g. Smoothness, wrinkles, melanin levels, transepidermal water loss, and facial epidermis moisture content were measured by a research assistant against both sides of the subjects' faces before (Before), 2 weeks after (After 2weeks), and 4 weeks after (After 4 weeks) the application of each cream was started.

Smoothness and wrinkles were measured using a VISIOSCAN (registered trademark) VC98 (manufactured by Courage +Khazaka electronic GmbH).

Melanin levels, transepidermal water loss, and facial epidermis moisture content were measured using CUTOMETER (registered trademark) manufactured by Courage+Khazaka electronic GmbH).

### <Statistical Processing>

Statistical analysis of data was performed using SPSS version 17.0 (SPSS Co., Ltd., Bangkok, Thailand). A paired t-test (p<0.05) was used to compare the test group (the side to whic0h the test cream was applied) and the control group (the side to which the control cream was applied).

### <Results>

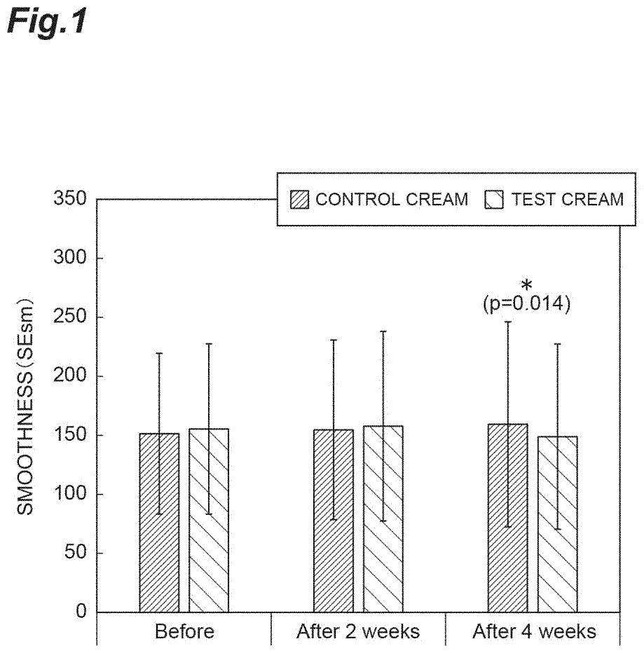

FIG. 1 is a graph showing evaluation results of smoothness (SEsm). A smaller SEsm value means smoother skin. Before the start of application of each cream (Before), there was no significant difference in smoothness (SEsm), but at 4 weeks after the start of application (After 4 weeks), the SEsm value (148.85±78.46) of the test cream-applied facial skin was significantly lower than the SEsm value (159.32±86.88) of the control cream-applied facial skin (p=0.014). That is, the test cream-applied facial skin became smoother than the control cream-applied facial skin.

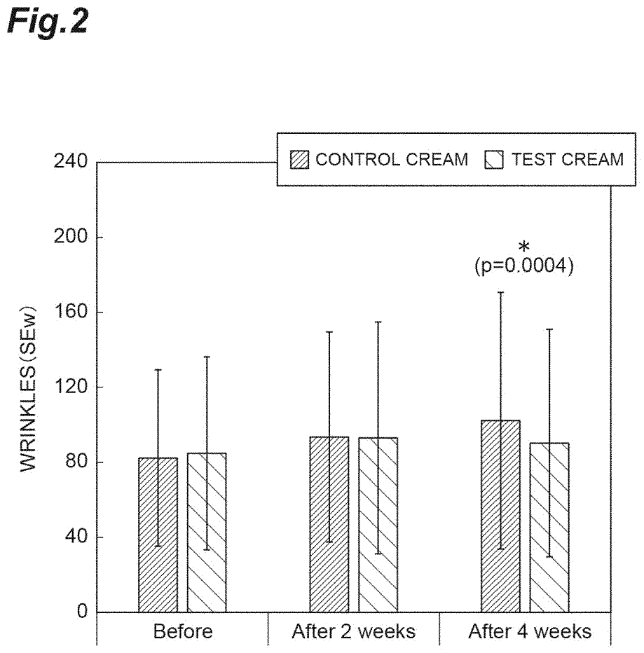

FIG. 2 is a graph showing evaluation results of wrinkles (SEw). A smaller SEw value means less wrinkles. Before the start of application of each cream (Before), there was no significant difference in wrinkles (SEw), but at 4 weeks after the start of application (After 4 weeks), the SEw value (90.26 ±60.68) of the test cream-applied facial skin was significantly lower than the SEw value (102.27 ±68.46) of the control cream-applied facial skin (p=0.0004). That is, the test cream-applied facial skin had less wrinkles than the control cream-applied facial skin.

These results show that the application of the bagasse-derived polyphenol composition for 4 weeks increased skin smoothness and reduced wrinkles.

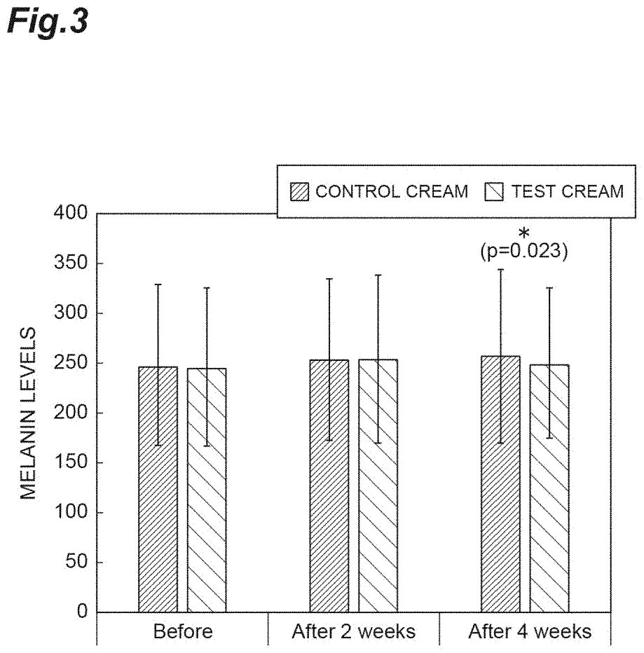

FIG. 3 is a graph showing evaluation results of melanin levels. Higher melanin levels mean more pigmentation. Before the start of application of each cream (Before), there was no significant difference in melanin levels, but at 4 weeks after the start of application (After 4 weeks), the melanin levels (249.44 ±75.46) of the test cream-applied facial skin was significantly lower than the melanin levels (257.02 ±87.45) of the control cream-applied facial skin (p=0.023). That is, the test cream reduced skin pigmentation.

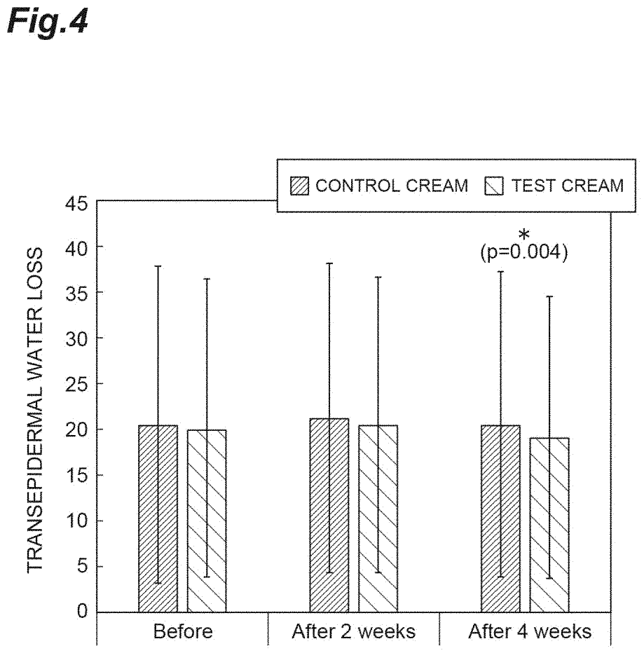

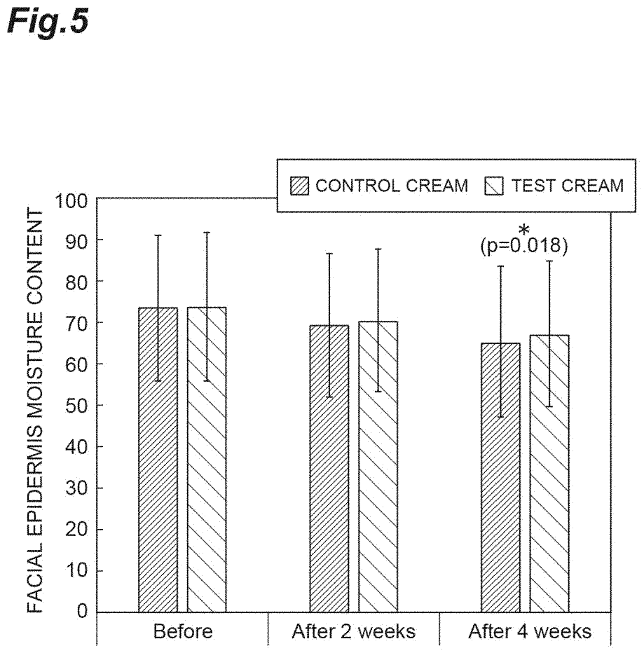

FIG. 4 is a graph showing evaluation results of transepidermal water loss. FIG. 5 is a graph showing evaluation results of facial epidermis moisture content. Low transepidermal water loss and high facial epidermis moisture content mean more hydrated skin (moist skin). Before the start of application of each cream (Before), there was no significant difference in transepidermal water loss, but at 4 weeks after the start of application (After 4 weeks), the transepidermal water loss (19.07±15.53) of the test cream-applied facial skin was significantly lower than the transepidermal water loss (20.51±16.81) of the control cream-applied facial skin (p=0.004). Before the start of application of each cream (Before), there was no significant difference in facial epidermis moisture content, but at 4 weeks after the start of application (After 4 weeks), the facial epidermis moisture content (67.28±18.21) of the test cream-applied facial skin was significantly higher than the facial epidermis moisture content (65.27±17.94) of the control cream-applied facial skin (p=0.018). That is, the test cream increased the skin moisture content.

These results show that the application of the bagasse-derived polyphenol composition for 4 weeks reduced skin pigmentation and increased the skin moisture content.

No skin reactions (rashes, edema, papules, and itching) were reported by the subjects. That is, it can be seen that each cream can be applied safely.

1.11. (canceled)

12. A method for improving skin condition, the method comprising:
administering an effective amount of an agent for improving skin condition to a subject in need thereof,
wherein the agent comprises a bagasse-derived polyphenol composition as an active ingredient.

13. The method according to claim 12, wherein the bagasse-derived polyphenol composition is obtained through a method including:
pretreating bagasse using at least one alkaline solution selected from the group consisting of an aqueous sodium hydroxide solution, an aqueous potassium hydroxide solution, and an aqueous ammonia solution to obtain a pretreatment solution;
adjusting a pH of the pretreatment solution to be acidic with hydrochloric acid, and then filtering the pretreatment solution to recover a filtrate; and
passing the filtrate through a column filled with an aromatic synthetic adsorbent and eluting a component adsorbed onto the above-described aromatic synthetic adsorbent with a mixed solvent of ethanol and water to obtain an eluted fraction as the polyphenol composition.

14. The method according to claim 13, wherein a temperature of the alkaline solution is 60° C. to 100° C.

15. The method according to claim 13, wherein the alkaline solution is an aqueous sodium hydroxide solution.

16. The method according to claim 15, wherein a concentration of the aqueous sodium hydroxide solution is 0.1 to 1.0% by mass.

17. The method according to claim 13, wherein the aromatic synthetic adsorbent is one consisting of a styrene-divinylbenzene resin.

18. The method according to claim 12, wherein the improving skin condition includes at least one selected from the group consisting of improving skin smoothness, reducing skin wrinkles, suppressing skin pigmentation, and increasing skin moisture content.

19. The method according to claim 12, wherein the agent further comprises at least one selected from the group consisting of oil agents, surfactants, moisturizing components, thickener, powders, inorganic pigments, organic dyes, organic powders, ultraviolet absorbers, lower alcohols, vitamins, proteins, and lipids.

20. The method according to claim 12, wherein the agent is in a form of solid, liquid or paste.

21. The method according to claim 12, wherein the subject is human.

22. The method according to claim 12, wherein the agent is applied to skin.

23. The method according to claim 22, wherein the application amount per day of the bagasse-derived polyphenol composition is between 1 ng/cm² and 100 ng/cm².
